(19) United States
(12) Patent Application Publication
(10) Pub. No.: US 2014/0255269 A1
(43) Pub. Date: Sep. 11, 2014

(54) SANITIZING DEVICE FOR TOILET SEAT

(71) Applicant: Paula Kelly, Ellenton, FL (US)

(72) Inventor: Paula Kelly, Ellenton, FL (US)

(21) Appl. No.: 14/196,598

(22) Filed: Mar. 4, 2014

Related U.S. Application Data
(60) Provisional application No. 61/775,827, filed on Mar. 11, 2013.

(51) Int. Cl. A61L 2/18

(52) U.S. Cl. CPC A61L 2/18
USPC 422/292

(57) ABSTRACT

A sanitizing device having a first tubular housing and a second tubular housing is provided. The first and second tubular housing are coupled together, and are disposed along a common axis when coupled together. The first tubular housing includes a sanitizing member. The sanitizing member may be formed of any material configured to apply a solution, such as a moist paper material treated with a disinfectant. The second tubular housing includes a drying member. The drying member may be from any material known to absorb moisture such as a sponge, a cloth, disposable paper or a synthetic material. The drying member may be further treated with a fragrance.

### CROSS-REFERENCE TO RELATED APPLICATIONS

This application claims priority of U.S. Provisional Application No. 61/775,827 filed on Mar. 11, 2013, the contents of which are incorporated herein by reference in their entirety.

### FIELD OF THE INVENTION

The invention relates to sanitizing device for sanitizing surfaces available for public use. More particularly the sanitizing device is a portable device having a first tubular housing and a second tubular housing. The first tubular housing includes a sanitizing member, and the second tubular housing includes a drying member.

### BACKGROUND OF THE INVENTION

For many people it is frequently necessary to use toilets in public buildings and businesses which are used by many other people. Frequently there is concern about the sanitary condition of the toilet seats in such facilities. Accordingly, thin paper toilet seat covers are provided. These covers are disposable. However, if the toilet seat is wet, moisture quickly soaks through the thin paper cover. Additionally, the covers are hard to use and dispose of.

### SUMMARY OF THE INVENTION AND ADVANTAGES

A sanitizing device having a first tubular housing and a second tubular housing is provided. The first and second tubular housing are coupled together, and are disposed along a common axis when coupled together. The first tubular housing includes a sanitizing member. The sanitizing member may be formed of any material configured to apply a solution such as a moist paper material treated with a disinfectant. The second tubular housing includes a drying member. The drying member may be a cloth or a disposable paper material configured to absorb moisture. The drying member may be a swab, and the swab may be made from any material known to absorb moisture such as a sponge, paper, cotton or a synthetic material. The drying member may be further treated with a fragrance.

In a first preferred embodiment, the first tubular housing and the second tubular housing each include a distal end opposite an open end. The first and second tubular housing are configured to be seated together. For example, the first tubular housing may include a recess portion formed at the open end, and the second tubular housing may include an insertion portion formed at the open end. The insertion portion is dimensioned to fit within the recess portion so as to seat the first tubular housing and second tubular housing together. Accordingly, the first and second tubular housing may be coupled together and slid away from each other for use.

In the first preferred embodiment, the sanitizing device includes a pair of disks. One disk is slidably disposed within the first tubular housing and the other disk is slidably disposed within the second tubular housing. The drying member and the sanitizing member are disposed within the respective first and second tubular housing between the pair of disks. The disks may be pushed towards the open end so as to push the drying or sanitizing member out of the respective first and second tubular housing and avail the drying member and sanitizing member for use.

In a second preferred embodiment, the sanitizing device includes an outer tubular housing. The outer tubular housing includes a pair of open ends. The first tubular housing and the second tubular housing are dimensioned to fit slidingly within the outer tubular housing. The first tubular housing and the second tubular housing are slidable with respect to each other within the outer tubular housing along a common axis.

In a third preferred embodiment the first and second tubular housing are coupled together at proximate ends so as to form a unitary unit. The sanitizing device includes a partition disposed between the first tubular housing and a second tubular housing. The first tubular housing is separable from the second tubular housing along the partition.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a first preferred embodiment of the sanitizing device showing the first tubular housing coupled to the second tubular housing;

FIG. 2 is a perspective view of the sanitizing device of FIG. 1, showing the recessed portion of an open end and the insertion portion of an open end of the first and second tubular housing;

FIG. 3 is a perspective view of the sanitizing device of FIG. 2, showing the drying member and sanitizing member pushed forward of a corresponding open end of the respective first and second tubular housing;

FIG. 4 is a cross sectional view of the sanitizing device of FIG. 2 taken along line 4-4;

FIG. 5 is a perspective view of the sanitizing device of the second preferred embodiment in a package with the elongated member being shown in broken lines;

FIG. 6 is a perspective view of the elongated member of FIG. 5;

FIG. 7 is a top down view of the elongated member of FIG. 6 showing the slot; and

FIG. 8 is a side view of the third preferred embodiment of the sanitizing device showing the partition.

### DESCRIPTION OF THE INVENTION

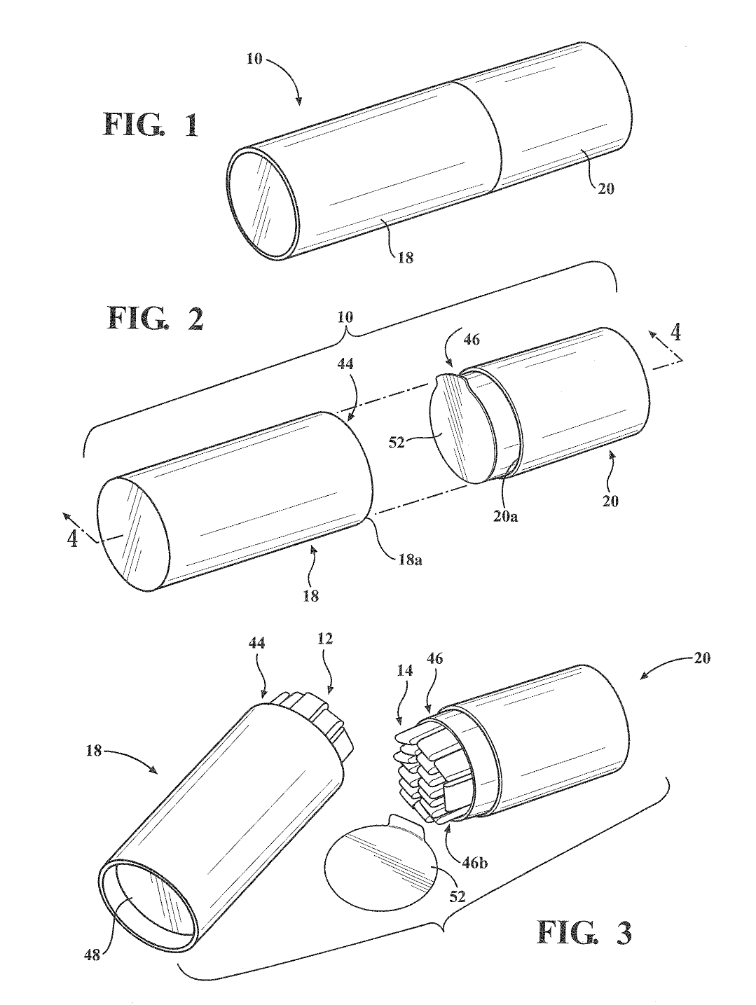

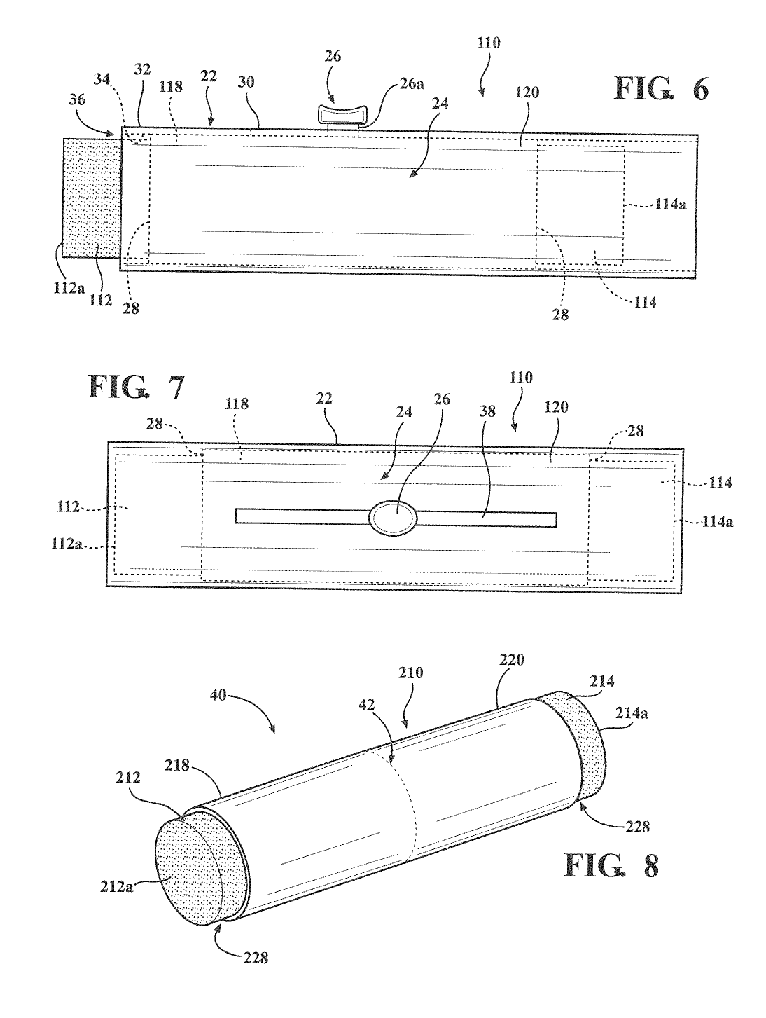

As shown in Figures, a novel sanitizing device 10 includes a sanitizing member 12 and drying member 14 which may be provided in an airtight package 16. The sanitizing device 10 may be dimensioned so as to be held within a user's hand. The sanitizing device may be sold off the shelf at a store or found within a vending machine. The sanitizing device 10 includes a first tubular housing 18 and a second tubular housing 20. FIGS. 1-4 show the first and second tubular housing 18, 20 formed as separate pieces, whereas FIGS. 5-8 illustrate that first and second tubular housing 18, 20 may be formed as a single unit. The first tubular housing 18 includes the sanitizing member 12, and the second tubular housing 20 includes the drying member 14.

With reference now to FIGS. 1-4, a first preferred embodiment of a sanitizing device 10 is provided. The first tubular housing 18 and the second tubular housing 20 each include a distal end opposite an open end 18a, 20a. The sanitizing device 10 includes a recess portion 44 and an insertion portion 46. The insertion portion 46 is dimensioned to fit within the recess portion 44 so as to couple the first tubular housing 18 and second tubular housing 20 together.

Though FIGS. 1-4 show the recess portion 44 formed on the second tubular housing 20, and the insertion portion 46 formed on the first tubular housing 18, it should be appreciated that the insertion portion 46 may be formed on the second tubular housing 20 and the recess portion 44 may be formed on the first tubular housing 18. Thus, one of either the first tubular housing 18 or the second tubular housing 20 includes the recess portion 44 formed at the open ends 20a, 20b, and the other of either the first tubular housing 18 or the second tubular housing 20 includes the insertion portion 46 formed at the open end 20a, 20b.

The first tubular housing 18 may hold the sanitizing member 12 and the second tubular housing 20 may hold the drying member 14. The free ends of the tubular portions are open so to provide access to disks 48 which are pushed to expose the sanitizing member 12 and drying member 14 when the two tubular housings 18, 20 are separated, as shown in FIG. 3. The first and second tubular housings 18, 20 may be of any cross sectioned shape but in the preferred embodiment, the tubular housings 18, 20 are shown with a circular cross section. The first tubular housing 18 has an insertion portion 46 which is formed to be snug within a recess portion 44 of the second tubular housing 20. The recess portion 44 includes a step 48 extending radially inward from an inner wall surface. The insertion portion 46 includes an outer wall 46a dimensioned to snugly fit within the inner wall 44a surface of the recess portion 44. A distal peripheral edge of the insertion portion 46 is configured to abut against the step 48 of the recess portion 44 so as to seat the first tubular housing 18 with the second tubular housing 20.

The free end of the first tubular housing 18 and second tubular housing 20 include a second opening 18b, 20b disposed at the respective distal ends. Each of the second openings 18b, 20b includes a stop 50. The stop 50 may be a flange which extends radially inwardly from the inner surface of the second opening 18b, 20b. The flange extends inwardly and is dimensioned to form a second opening 18b, 20b large enough to accept a person's finger.

The sanitizing device 10 is formed by interconnected first and second tubular housings 18, 20. The disks 48 have a dimension to be slidingly received within a corresponding inner surface of a respective first and second tubular housing 18, 20. The disks 48 are positioned at the end the first and second tubular housings 18, 20, and the sanitizing member 12 and drying member 14 are disposed between the disks 48 when the first tubular housing 18 is coupled with the second tubular housing 20.

The sanitizing member 12 is shown mounted within the first tubular housing 18 so that pushing the disk 48 with a finger will push the sanitizing member 12 from the insertion end of the first tubular housing 18 making it available for use as shown in FIG. 7.

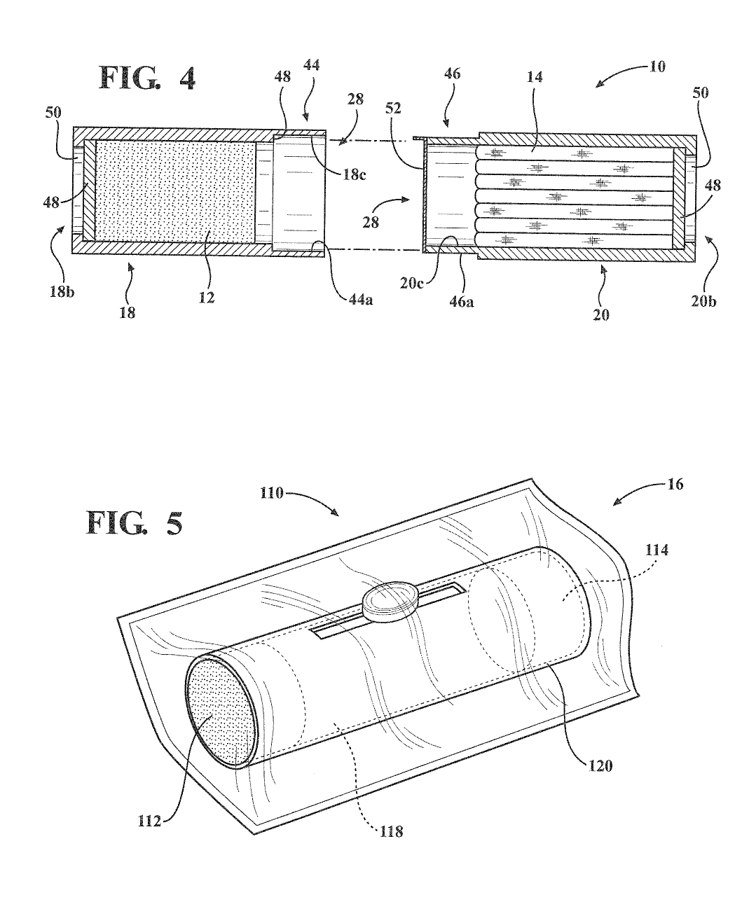

A seal 52 may be mounted across an opening 46b of the insertion portion 46 of the first tubular housing 18 to keep the compartment defined therein sealed to maintain the moisture in the sanitizing member 12. The seal 52 may be plastic or foil sheeting. The sanitizing member 12, such as a wad of moist paper may be enclosed in individual packets, a plurality of which are held within the first tubular housing 18 as shown in FIGS. 3, and 4. Alternatively, the sanitizing member may be a swab mounted to one end of the inner elongated member 24 (not shown). In such an embodiment, the sanitizing swab is enclosed by the seal and may be pushed out of the first tubular housing 18 via the disk 48. The sanitizing member 112 may be formed of any suitable material configured to applying a solution such as a sanitizing liquid or a disinfectant. An acceptable substrate is the paper material used in products like towelettes.

The second tubular housing 20 has an inner chamber 20c for receiving the drying swab or sponge and the disk 48 is mounted at the second opening 20b of the second tubular housing 20. The swab may be made from any material known to absorb moisture such as a sponge, paper, cotton or a synthetic material. The disk 48 is retained in the tube by a stop 50 flange as in the first tubular housing 18.

In order to use the sanitizing device 10 the first and second tubular housings 18, 20 are separated and the seal is pulled from the first tubular housing 18. The disk 48 is pressed against the sanitizing member 12 until the end extends outwardly from the insertion portion 46. (FIG. 8) The sanitizing member 12 then may be used by holding the outer surface of the first tubular housing 18 to clean a toilet seat. After use the first tubular housing 18 is discarded and the drying swab is pushed out of the second tubular housing 20 for use in drying. The first and second tubular housings 18, 20 may be made from biodegradable materials so that they can be flushed for disposal.

With reference now to FIGS. 5-7 a second preferred embodiment of the sanitizing device 110 is provided wherein like elements are referenced by the same number increased by 100. The first tubular housing 118 is fixedly coupled to the second tubular housing 120 so as to form an elongated inner member 24. The distal ends of the first and second tubular housings 118, 120 are open.

The sanitizing device 110 includes an outer tubular housing 22. The elongated inner member 24 is slidably disposed within the outer tubular housing 22. The outer tubular housing 22 is a generally cylindrical tube having a pair of opposed opposite ends. Each of the opposite ends has an open end 28. The outer tubular housing 22 has a wall 30 with an outer surface 32 and an inner surface 34 defining a cavity 36. An axially aligned slot 38 extends through the wall 30 of the outer tubular housing 22 to the inner cavity 36. The slot 38 is formed to accept a stem 26a of the handle 26. The stem 26a extends through the slot 38 to the inner elongated member 24, and is fixed thereto. The outer tubular housing 22 may be formed of a biodegradable material such as thick paper or cardboard which can be flushed down the toilet.

The inner elongated member 24 is generally cylindrical and formed to slide within the inner wall 30 of the outer tubular housing 22. The sanitizing member 112, such as a wad 112a of moist paper, is mounted to one end of the inner elongated member 24 and is axially outwardly from the end of the inner elongated member 24. The sanitizing member 112 may be formed of suitable material for applying a sanitizing liquid. An acceptable substrate is the paper material used in products like towelettes. The drying member 114 such as a wad 114a of absorbent paper or a sponge and may be mounted within a cavity 36 formed at the other end of the inner elongated member 24. The wad 114a may be made from any material known to absorb moisture such as a sponge, paper, cotton or a synthetic material.

The inner elongated member 24 with the axially and outwardly drying member 114 and sanitizing member 112 are generally the same length as the outer tubular housing 22 and is received within the outer tubular housing 22 so that the sanitizing and drying members 114 are maintained just inside the outer tubular housing 22. It should be appreciated that the inner elongated member 24 may be dimensioned so that a portion of either the sanitizing member 112 or the drying member 114 extends axially beyond an end of the outer tubular housing 22.

The stem 26a of the handle 26 is mounted through the slot 38 and into the inner elongated member 24. The handle 26 may have an enlarged outer end for grasping by the user. The inner elongated member 24 with the sanitizing member 112 and drying member 114 may be contained fully within the outer tubular housing 22 during shipment. Alternatively, the sanitizing member 112 may extend outside of the outer tubular housing 22 during shipment. In such an embodiment, the entire sanitizing device is wrapped in a seal tight package 16.

If the sanitizing member 112 of the inner elongated member 24 is mounted within the outer tubular housing 22, the handle 26 may be pushed in the direction of the sanitizing member 112 to move the inner elongated member 24 and sanitizing member 112 out of the outer tubular housing 22 before use in sanitizing the toilet seat. Movement of the handle 26 in the opposite direction results in retraction of the sanitizing member 112 within the outer tubular housing 22 and an extension of the drying member 114 from the outer tubular housing 22. The outer tubular housing 22 is then inverted and the toilet seat may be dried with the drying member 14. A latex glove may be included in the package 16 for use in manipulating the sanitizing device and handling the toilet seat.

It should be appreciated that the handle 26 shown is illustrative and not limiting to the scope of the invention, for instance the sanitizing device 110 may include a sleeve (not shown) or band (not shown) that encircles the outer tubular housing 22 and slides on the outer tubular housing 22. The sleeve or band may include a stem (not shown) connecting the inner surface of the sleeve or band to the inner elongated member 24. The stem is movable along a slot (not shown) so as to move the respective ends of the elongated member in and out of the outer tubular housing 22.

It is important that the sanitizing member 112 be stored in an airtight package so that it does not dry out prematurely. Accordingly, the device 110 may be delivered in a package 16. The package 16 may be an airtight pouch or packaging material. The packaging material may have a foil laminate to prevent migration of air into the package. Alternatively, seals may be positioned over the ends of the outer tubular housing 22 so that no air gets into the outer tubular housing 22. After use the sanitizing device 110 is discarded.

As shown in FIG. 8, a third preferred embodiment of the sanitizing device is provided wherein like elements are referenced by the same member increased by 200. The sanitizing device 210 includes a first tubular housing 218 and a second tubular housing 220 formed together as a single tube unit 40. The outer surface of the single tube unit 40 is used as a handle with the drying member 214 and sanitizing member 212 disposed on opposite ends of the single unit 40. For instance, the sanitizing device 210 includes a drying swab 214a or sponge extending from one end and a sanitizing swab 212a extending from the other end. The sanitizing device 210 includes a partition 42 between the first and second tubular housing so as to allow one to break away from the other. In one embodiment the partition 42 is a plurality of serrations which extend circumferentially around the middle of the single tube unit to break off the sanitizing member 212 after use and permit sanitizing member 212 to be discarded. The remaining portion, the drying member 214 is then used to dry the toilet seat.

Obviously, many modifications and variations of the present invention are possible in light of the above teachings and may be practiced otherwise than as specifically described while within the scope of the appended claims.

What is claimed is:

1. A sanitizing device for sanitizing a surface, the sanitizing device comprising:
a first tubular housing and a second tubular housing, the first and second tubular housing coupled together, wherein the first tubular housing includes a sanitizing member, and the second tubular housing includes a drying member.

2. The sanitizing device as set forth in claim 1, wherein the first tubular housing and the second tubular housing each include a distal end opposite an open end, wherein one of either the first tubular housing or the second tubular housing includes a recess portion formed at the open end, and the other of either the first tubular housing or the second tubular housing includes an insertion portion formed at the open end, the insertion portion dimensioned to fit within the recess portion so as to couple the first tubular housing and second tubular housing together.

3. The sanitizing device as set forth in claim 2, further including a pair of disks, one of the pair of disks is slidably disposed within the first tubular housing and the other of the pair of disks is slidably disposed within the second tubular housing, wherein the drying member and the sanitizing member are disposed between the pair of disks.

4. The sanitizing device as set forth in claim 3, wherein the first tubular housing and a second tubular housing each include a second opening disposed at the respective distal ends, the second opening having a stop the second opening providing access the a respective disk in the pair of disks so as to allow the user to push the disk towards a respective opening end or insertion portion.

5. The sanitizing device as set forth in claim 3, further including a seal disposed over the open end of the insertion portion so as to seal the sanitizing member within the second tubular housing.

6. The sanitizing device as set forth in claim 1, further including an outer tubular housing, the outer tubular housing having a pair of open ends, the first tubular housing and the second tubular housing dimensioned to fit slidingly within the outer tubular housing.

7. The sanitizing device as set forth in claim 6, wherein the first tubular housing is fixedly coupled to the second tubular housing so as to form an elongated inner member, the elongated inner member is slidably disposed within the outer tubular housing, the distal ends of the first and second tubular housings are open.

8. The sanitizing device as set forth in claim 7, wherein the sanitizing member is on one end of the elongated inner member, and the drying member is disposed the opposite end of the elongated inner member.

9. The sanitizing device as set forth in claim 8, further including a handle, a portion of the handle is exposed on the outer tubular housing, the handle dimensioned to be gripped by a user.

10. The sanitizing device as set forth in claim 9, wherein outer tubular housing includes a slot the handle disposed within the slot, the handle fixedly mounted to the elongated member so as to axially slide the elongated member with respect to the outer tubular housing.

11. The sanitizing device as set forth in claim 1, further including a partition disposed between the first tubular housing and a second tubular housing, the first tubular housing separable from the second tubular housing along the partition.

12. A sanitizing device for sanitizing a surface, the sanitizing device comprising:
a first tubular housing and a second tubular housing, the first and second tubular housing coupled together, wherein the first tubular housing includes a sanitizing member, and the second tubular housing includes a drying member, wherein the first tubular housing and the second tubular housing each include a distal end opposite an open end, wherein one of either the first tubular housing or the second tubular housing includes a recess portion formed at the open end, and the other of either the first tubular housing or the second tubular housing includes an insertion portion formed at the open end, the insertion portion dimensioned to fit within the recess portion so as to couple the first tubular housing and second tubular housing together.

13. The sanitizing device as set forth in claim 12, further including a pair of disks, one of the pair of disks is slidably disposed within the first tubular housing and the other of the pair of disks is slidably disposed within the second tubular housing, wherein the drying member and the sanitizing member are disposed between the pair of disks.

14. The sanitizing device as set forth in claim 13, wherein the first tubular housing and a second tubular housing each include a second opening disposed at the respective distal ends, the second opening having a stop the second opening providing access the a respective disk in the pair of disks so as to allow the user to push the disk towards a respective opening end or insertion portion.

15. The sanitizing device as set forth in claim 13, further including a seal disposed over the open end of the insertion portion so as to seal the sanitizing member within the second tubular housing.
